(19) United States
(12) Patent Application Publication
(10) Pub. No.: US 2006/0153935 A1
(43) Pub. Date: Jul. 13, 2006

(54) Stabilizing salicylate compositions and method of preparation for oral and topical use

(76) Inventor: Natalie Blahut, Tallahassee, FL (US)

Correspondence Address: Ralph A. Dowell of DOWELL & DOWELL P.C., 2111 Eisenhower Ave, Suite 406, Alexandria, VA, 22314, US

(21) Appl. No.: 11/327,380

(22) Filed: Jan. 9, 2006

Related U.S. Application Data
(60) Provisional application No. 60/641,748, filed on Jan. 7, 2005.
(60) Provisional application No. 60/696,520, filed on Jul. 6, 2005.

(51) Int. Cl. A61K 31/60; A61K 36/736; A61K 36/886; A61K 36/53; A61K 36/28

(52) U.S. Cl. 424/735; 514/165; 424/745; 424/764; 424/744

(57) ABSTRACT

Salicylic acid or acetylsalicylic acid are combined with citric acid and then mixed with a concentrated potassium hydroxide solution to form a stable compound of either potassium salicylic acid or potassium acetylsalicylic acid that can be used to make lotions, creams, gels or ointments for topical formulations or mixed to form a sports, health or therapeutic drinks.

### CROSS REFERENCING OF PRIOR APPLICATIONS

This application claims benefit of the earlier filing date of U.S. Provisional Application 60/641,748, filed on Jan. 7, 2005 and U.S. Provisional Application 60/696,250, filed on Jul. 6, 2005 and is incorporated by reference herein.

### BACKGROUND

Plant-based salicylic acid is gaining recognition as a powerful phyto-chemical, similar to ascorbic acid, a flavonoid. Salicylic acid is generated by plants for their defense against disease. The production of salicylic acid is greatly reduced in plants when pesticides are used, as pesticides inhibit the plants' need to protect themselves. Without this intervention the amount of salicylic acid is very high in a variety of plants including several varieties of berries which are among the best-known natural sources of salicylic acid. Where these berries are abundant and freely eaten, the level of salicylic acid in the surrounding population is often as high as it is in people on traditional aspirin therapy. Salicylic acid obtained from food is found in a complex of other nutrients which together make the salicylic acid soothing.

Pure synthetic salicylic acid is very acidic, not completely water-soluble and is difficult to alkalinize with other substances. This is a continuing problem for aspirin that adding buffers does not solve. Salicylic acid and derivatives like acetylsalicylic acid (aspirin) are limited in the amounts which can be used either orally or topically due to these buffering problems even as new uses for salicylates are discovered. Modifying synthetic salicylates so that their desirable characteristics are more readily available and their aversive ones are eliminated has been an elusive research goal for the last century.

Reformulating synthetic salicylic acid to increase its available uses is an obtainable goal. If properly reformulated, the synthetic salicylic acid can emulate the characteristics of salicylic acid in its natural state and can be abundantly produced for promoting health and healing in plants, humans and animals. Once synthetic salicylic acid is throughly stabilized and freed of its excess acidity, it uses broaden. Low acid and alkaline salicylates such as acetylsalicylic acid become possible as well as other salicylates which do not thin blood. Salicylates having a wider range of pH values can be used for topical applications, for health drinks, as preservatives, as flavorings, for flea and tick control, as a dental remedy, or used in combination with different emollients. The salicylates are mild even at highest concentrations for external transdermal use.

Over the past century the most compelling reason for improving aspirin was the need to use it freely, in higher doses and long-term if necessary. The salicylates described here found their first test in an 8-year bout with disabling Fibromyalgia while they were being developed. Using them liberally, orally and topically not only reduced the symptoms of Fibromyalgia to nil but an unexpected outcome of this dosing was the disappearance of other symptoms, such as neck and muscle cramps, spasms, headaches, stress, etc., suggesting that such dosing also promoted health and well-being and prevented further illness. Fibromyalgia is increasingly being described as an inflammatory rheumatoid process, just as inflammation is being recognized as a basic defensive physiological process underlying all disease (Time, 2/04 The Fires Within); furthermore, aspirin, imperfect as it is, is still the oldest and most universal drug for treating inflammation. The new salicylates broaden aspirin's reach through many formulations which are more powerful at the same time that they are more benign.

The customary oral dosage of a salicylate need not increase for higher dosing, although it is an option if the need for adequate hydration is observed and ingestion is gradual enough to allow for feedback from the body as the salicylate becomes readily and noticeably available. Higher dosing can also be achieved by decreasing the time between dosing or sipping frequently as needed, as well as by concurrent oral and topical dosing as needed, which is the most effective. This runs counter to a fixed schedule of taking medications which sometimes work against the body's natural order and aversive effects may not be apparent before becoming critical. Ongoing feedback from the body in the long run provides a more gradual but accurate and steady indicator for continued dosing. In addition, when the salicylate is taken orally, pain may sometimes be noted in an extremity which then benefits from additional topical applications.

### SUMMARY

Salicylates can be stabilized with high alkaline potassium hydroxide solution and citric acid for oral and topical use. The effect of a concentrated alkaline solution on a salicylate can best be described as corrosive, totally dissolving the salicylic acid or acetylsalicylic acid in a supersaturated highly alkaline state which remains liquid only while hot. Both the more powerful potassium hydroxide solution and the citric acid, which also acts as a preservative, make possible salicylates which can be produced in more varied physical forms which significantly extend their uses; some of these may not need dehydration or can be dehydrated without specialized equipment.

Broadening the basic physical forms of potassium salicylic acid and potassium acetylsalicylic acid by the addition of citric acid increases their uses. The addition of citric acid to the salicylate formulas: 1) requires the potassium hydroxide stabilizing solution to be more alkaline and therefore more powerful; 2) acts as a preservative; 3) adds flavoring solids for oral uses; 4) allows the salicylates to be made in more forms, such as powdered, liquid or other forms because of increased stability and resistance to breakdown of the resultant product; 5) eliminates the need for the extra step of dehydration for some formulas. The varied forms have a wider range of applications and make it easy to custom-make formulas for desired outcomes, such as health drinks, flea and tick control for pets as well as other formulations described herein.

TABLE 1
Formulas for Potassium Salicylic Acid and Potassium Acetylsalicylic Acid

| | | | | | | K, SA, or ASA Yield from | pH of final |
|---|---|---|---|---|---|---|---|
| | Formula | KOH | SA | ASA | CA | production of KSA or KASA | product |
| 1 | Alkaline KSA | 40-42 gm | 76 gm | | 5 gm | 132-137 mgK/628-618 mgSA | 7.19-9.24 |
| 2 | Alkaline KASA | 43-44-45 gm | | 55 gm | 5 gm | 167-169 mgK/534-529 mgASA | 7.25-8.20-9.08 |
| 3 | Acid KSA | 35-37 gm | 76 gm | | 5 gm | 120-126 mgK/655-644 mgSA | 5.95-6.48 |
| 4 | Acid KASA | 39-41 gm | | 55 gm | 5 gm | 158-162 mgK/556-545 mgASA | 6.12-6.97 |
| 5 | Acid KSA | 39 gm | 76 gm | | 15 gm | 120 mgK/585 mgSA | 5.22 |
| 6 | Acid KASA | 39 gm | | 55 gm | 15 gm | 143 mgK/504 mgASA | 5.08 |

KOH = Potassium Hydroxide
ASA = Acetylsalicylic Acid
KSA = Potassium Salicylic Acid
SA = Salicylic Acid
CA = Citric Acid
KASA = Potassium Acetylsalicylic Acid

In Table 1 all ingredients in the potassium salicylic acid or potassium acetylsalicylic acid formulations are flavoring agents, i.e., they are virtually noncaloric ingredients which are widely added to foods and beverages (except possibly acetylsalicylic acid) either primarily for flavoring or for other purposes and in addition add desirable flavors; they are of value in the oral dosing formulas and the drinks which may include any formula in the first four of the six sets of formulas. Usually the desirable pH range for alkaline salicylate dosing formulas is considered to be 8-10 to counteract excess acidity in the gastrointestinal tract; for these formulas, at least, this is too alkaline for taste; any pH just above the neutral 7, for example 7.19 for potassium salicylic acid and 7.25 for potassium acetylsalicylic acid in Table 1, may be preferred for taste, which can be further diluted for a still milder alkaline flavor, with the added benefit of increased hydration. For a slightly more preferred flavor, the acidic potassium salicylic acid with a pH of 6.21 or the acidic potassium acetylsalicylic acid with a pH of 6.12 can be used for dosing, without a noticeable difference in gastrointestinal benefit.

The acid formula potassium salicylic acid, pH 5.22 and potassium acetylsalicylic acid pH 5.08 are for topical use formulas, among other possible uses, with a most preferred pH of 4.5-5.0 for use as a micro-biocide compatible with mammalian skin but with an acceptable pH range up to about 6.5 for the final formulations; a pH less than 4.5 is too acidic for any added benefit. Using aloe vera juice, with its pH of ±4.00, as the carrier for topical formulations keeps its final pH in this most preferred pH range.

Salicylic acid and acetylsalicylic acid values in the potassium salicylic acid and potassium acetylsalicylic acid formulas listed in Table 1 are based on the milligrams of each in one gram of the dry product produced from the methods described. What may appear to be a low dose oral modified salicylic acid may actually have a significant and pleasant systemic effect because all the salicylic acid is available.

Method of Stabilizing Salicylic Acid and Acetylsalicylic Acid with Potassium Hydroxide (KOH) and Citric Acid: Using the formulas of Table 1 the potassium hydroxide is dissolved in a preheated, preferably glass, container in approximately ±75-150 ml of water (depending on the total amount of solids in the formula) that has been heated to a temperature approaching boiling to facilitate dissolution. The salicylic acid or acetylsalicylic acid and the citric acid are mixed dry then dissolved in the hot potassium hydroxide solution; this further increases the temperature of the resultant solution. If the mixture of potassium hydroxide, citric acid and either salicylic acid or acetylsalicylic acid begins to thicken, more hot water can slowly be added sparingly to the mixture. Supersaturated salicylate formulas become more difficult to make as the proportion of solids to liquids increases. A large volume of solid particles must combine with very little water and dissolve: it must then remain liquified, and therefore hot, for whatever step in the process is next, such as dehydration or combining with other ingredients, which are also usually liquid. A light drop in temperature, e.g., due to a pause in production or inadvertently dropping more salicylate in the hydroxide than it can dissolve quickly, may cause clumping. This clumping may be difficult to dissolve before resulting in a slight temperature drop which causes loss of liquidity. A hydroxide solution started with nearly boiling water using heated utensils and kept in gently bubbling state by stirring constantly while adding dry salicylic/citric acid or acetylsalicylic acid/citric acid in a gradual but steady stream, the chemical reaction of which also produces heat, all contribute to rapid dissolution. The higher pH in the earlier part of stabilization also facilitates the process, just as slowing down the chemical process may even bring it to a halt. Reheating may restore the formula but its quality may be called into question. The stabilized liquid potassium salicylic acid or potassium acetylsalicylic acid can then be used

full strength topically as is or with enhancements;

dehydrated to a powdered form;

diluted to form a liquid oral dosing formulation;

poured directly into topical or other usually acidic liquid formulations.

Method of Dehydrating a Supersaturated Salicylate Solution: An improvised primitive dehydration method was used to determine how well the supersaturated salicylic solutions dehydrated. The method produced thoroughly stabilized powdered salicylates with good keeping quality which dissolved easily, were benign and beneficial to the gastrointestinal tract, whether alkaline or acid and could be used in higher doses for longer periods of time and exceeded the performance of the original salicylates. It was found that the powdered salicylate form can be dehydrated by pouring the hot freshly stabilized liquid into thin layers on plates. The plates are preheated to about ±170° F.; much higher may scorch the formula, much lower temperatures result in poor and uneven dehydration. The plates are allowed to dry then scraped to remove the powdered product. Commercial systems available for dehydration include Niro® systems which use fluid bed dryers, airstream drying systems, flash dryers, spray drier absorbers or granulators, at least one of which has worked well with salicylates. Formulas 1 and 2 are mild powdered slightly alkaline salicylate dosing formulas which can be placed on the tongue with a small amount of liquid and dissolved without an adverse reaction; therefore, encapsulating the powder without further processing is preferred to other dosing forms on the market, such as hard pills and tablets. Dissolution of the powdered salicylate is quick and gentle. The powdered salicylate form can be packaged in individual doses, similar to other commercial formulas on the market, or packaged in bulk to be spooned out in amounts as desired for a variety of uses, e.g., for oral dosing, health drinks and foods (e.g., soups, puddings), etc., for human and animal use. Formulas 3 and 4 are mildly acidic liquid salicylates for preparing the liquid formulations for oral dosing. Use of these formulas calls into question the generally accepted notion that alkaline dosing salicylates are to be preferred over a mild slightly acid salicylate which can actually be enjoyed diluted in liquid form for oral use. The pH of the resultant product prepared by the different methods remains the same regardless if dehydrated or not. The salicylates are completely stabilized and the formulations prepared by all methods are indistinguishable one from another in their usage in side by side comparisons. Formulas in Table 1 are only examples of the many formulas that can be made for different uses; neither are uses given limited to single formula. Dosages in Table 1 are the grams of salicylic or acetylsalicylic acid found in one dry gram of potassium salicylate.

Topical formulations described herein consist of two active ingredients, potassium salicylic acid or potassium acetylsalicylic acid, added to a carrier liquid, preferably aloe vera juice, gel and/or water, usually with vegetable glycerin (vegetable glycerin as the more basic preferred enhancement) and guar gum. Vegetable glycerin soothes and hydrates the skin; while guar gum adds essential fatty acids to the skin and emulsifies and thickens the formulation. The aloe vera juice also soothes and enriches the skin. Alternatively, the active ingredients, potassium salicylic acid and potassium acetylsalicylic acid, may be added to any other suitable carrier and enhanced by other suitable ingredients. Pure lavender oil is most preferred as an additional enhancement, particularly for use on burns (where it may even be used alone with good results). Lavender oil adds additional anti-inflammatory, analgesic and anti-bacterial benefits to topical formulations. The concentration of salicylic acid or acetylsalicylic acid in a formulation may range from less than 1% to more than 30% for Formulas 5 and 6 (Table 1), which is produced in the process of stabilizing the salicylates, as described earlier. Undiluted, the most concentrated formulas may be applied short-term directly to unbroken skin, as for hot spots on dogs or blemishes in humans or to alleviate intense pain. Adding only diluted vegetable glycerin for hydration and to soothe the skin extends their use for longer periods and on more sensitive injury. At 4-6% salicylic acid or acetylsalicylic acid, the formulations find their widest range of uses, including alleviating pain and therefore becoming useful in the treatment of burns. The most preferred concentration of vegetable glycerin for these formulations is ±4-5%; to use less is to deprive the skin of its benefits, especially when using higher concentrations of salicylic acid or acetylsalicylic acid with their tendency to dry the skin. More than ±4-5% may make the formulation tacky. Guar gum thickens the formulation while it imparts its other benefits, so its concentration is tied to this feature. At low concentrations, it is watery but suitable for uses requiring less thickening, like applying a lotion to a furry animal. At medium concentration, it becomes a thick lotion with many uses; at slightly higher concentration it is a gel, preferred by many to the lotion, which may feel cold; and adding a little more guar gum makes it a thick gel, suitable for adhering to teeth and gums while treating and cleaning them, and even thicker gel can be nearly dried to a thin poultice strip for placing on gums. These values may differ widely as concentrations of solids in the liquids differ and as the proportions of ingredients in different formulations vary. Where tackiness is not a concern, e.g., in dental care and a high level of soothing is needed to offset the effects of a salicylate on mouth tissues, higher concentrations of both vegetable glycerin (10%) and guar gum are indicated; one to soothe, the other to shield the mouth tissues from the effects of salicylic acid and allow the active ingredients to work gradually as saliva dissolves the guar gum. Again the concentration of salicylic acid and acetylsalicylic acid in a formulation with the broadest range of uses is about 4-6%; below 4% is less effective for the uses described herein. Pain is more consistently relieved beginning at about 4% however, some pain and dermatological uses as well as flea and tick control require a salicylic acid or acetylsalicylic acid level of about 5-6% or even much higher to be effective. For deeper soft tissue injury, such as in the heel or ball of the foot, 8-10% salicylic is also more effective; if pain is not relieved sufficiently the gels of highest concentration work best and are applied several times a day, preferably aided by light massage. These formulations can be applied as frequently as needed.

### Formulas and Descriptions

Potassium salicylic acid and potassium acetylsalicylic acid formulas are divided into two groups: either for oral use or for topical use.

Oral dosing formulas can be encapsulated, dissolved or diluted in water, drinks or food and used to relieve pain or for any of the uses of aspirin, keeping in mind that salicylic acid formulas do not thin blood. Topical application is used as a transdermal delivery system for salicylates as when potassium salicylic acid or potassium acetylsalicylic acid lotion is applied to most of the body for systemic relief of an ailment. Such dosing, though unquantified, is moderate and beneficial in practice and can be repeated as often as needed. Oral and topical formulas can also combine when oral dosing leaves a peripheral area still in need of further dosing, e.g., only extremity is in pain, at which point local topical application is added and works well. Concurrent oral and topical dosing may be beneficial especially when high dosing is needed. All examples are single ways of making a formula, and should not be considered limiting.

### EXAMPLE 1

Oral salicylates: Dehydrated potassium salicylic acid or potassium acetylsalicylic acid for oral dosing is prepared by the described method of stabilizing salicylic and acetylsalicylic acid using Formulas 1 or 2 (Table 1) and dehydrated.

Any given salicylate formula can be dehydrated but if it is going to be used in an acidic liquid formula, dehydration may not be necessary. Dehydration and pH: Formulas with a pH above 7 are unstable in the presence of water for extended periods, therefore need moisture totally removed if not combined with acidic ingredients. Whether to dehydrate and how, therefore, depends on the end use of the given formula: Formulas 1 and 2 (Table 1) having a pH of 7+ require the usual more elaborate removal of moisture to ensure a stable dry salicylate for dosing. Formulas 3 and 4 (Table 1) are acidic enough to make into liquid formulations for oral dosing. One dry gram or 2.5 ml of liquid formulation for oral dosing is considered a dose; salicylic acid or acetylsalicylic acid values vary from formula to formula. Whether dehydrated or liquid, salicylates are equivalent in their pH and their uses in side by side comparisons.

### EXAMPLE 2

Liquid formulations for oral dosing: To formulate a liquid for oral dosing, additional preferably hot water is added gradually to the freshly stabilized liquid potassium salicylic acid or potassium acetylsalicylic acid of preferably slightly acid Formulas 3 or 4 (Table 1). The amount of water added should increase the total milliliters of liquid to the total number of grams of dry ingredients in the formula multiplied 2.5 times. As an example, 12 gms of dry ingredient multiplied by 2.5 increases the total milliliters of the formulation to equal 30 ml. The resultant product yields a dose which is equivalent to one gram of dehydrated salicylate in a 2.5 ml (one half teaspoon) dose. Salicylic acid and acetylsalicylic acid values vary from formula to formula. Variant of liquid formulation: freshly stabilized salicylate, cooled and undiluted, settles into a moist semisolid form and can be used at half strength for Formulas 3 and 4 (Table 1); which takes up half the space is easier to transport than liquid; and may be enhanced by one teaspoon lemon juice and Stevia powder, a noncaloric ingredient, used to sweeten and enhance, to make 250 ml of drink, with pH of 4.14 suitable for a cold or hot health beverage; and also blends well with orange juice; adding two teaspoons concentrated pure cherry juice (Tree of Life was used) with a teaspoon of lemon juice to make 250 ml of tasty fruit drink which contains about 30 calories.

### EXAMPLE 3

A sports/health/therapeutic drink: Adding one half teaspoon of liquid or one gram of dehydrated potassium salicylic acid or potassium acetylsalicylic acid and 2-3 (10-15 ml) teaspoons of lemon juice makes a slightly tart to tart tasty health or dosing drink with a pH above 3.0 with less than 1 gram of sugar, and can be obtained from either Formula 1 or 2 (Table 1). Less lemon juice is overwhelmed by the salicylate, while more is too tart (and 15 ml may be too tart) for a tasty drink. Tart lemon drinks are best used in moderation and are not for high dosing or heavy use in hydration. Additionally 2 teaspoons of concentrated cherry juice (Tree of Life was used), can be added to the above formula to render a pink lemonade drink with less than 3½ gms of sugar per 250 ml of drink to make a flavorful drink. A virtually non-caloric “fitness water” may be used for encouraging hydration with only a hint of flavor, in this case, lemon juice (½ teaspoon) and a half dose of the same liquid product to make 250 ml of drink. All oral dosing formulas and drinks may be enhanced with other suitable colors, textures (e.g., fizzes) or compatible nutrients for added appeal including frozen orange juice concentrate which blends well for a somewhat more caloric salicylate health drink.

### EXAMPLE 4

A topical lotion or gel with 4%-6% salicylic acid or acetylsalicylic acid has the broadest range of uses. This is the point above which in addition to dermatological uses, the formula includes amelioration of pain and is well above current over-the-counter (OTC) levels which range up to approximately 2.5% active ingredient. At 4-6% it is the salicylic acid concentration which also relieves pain first when used on burns. The active ingredients may be added to any other suitable carrier lotion, gel, ointment, etc.

Method of Blending Topical Formulations: To blend the ingredients comprising the topical gels or lotions requires that the hot freshly stabilized potassium salicylate remains liquid at the same time that it needs to cool down in order to be combined with a cold-pressed aloe vera juice or gel, which needs to remain cool. Since the stabilizing process is kept close to boiling and supersaturated, before cooling down it needs to be diluted with hot water first so that it remains liquid while cooling down for blending. The glycerin must be diluted separately in some water, then added to the cooler salicylate. Aloe vera juice or gel is then blended in, together with other enhancements, such as lavender or other essential oil to complete the quantity of gel or lotion being made. Guar gum is usually added last. The amount of this emulsifier used for the desired consistency varies with the kinds, amounts and concentrations of ingredients in the formulations. The most preferred topical formulation is a thick gel which uses between 1½ and 2% guar gum. One percent makes a light lotion, sometimes watery; special uses, like dental remedies require nearly dry lotions and require higher concentrations, but vary widely according to the formulations. The personal care uses of the topical lotion or gels proliferate to include deodorants, washes, microbiocides, lubricants, anti-burn gels/lotions, anti-itch gels/lotions, or used for muscle spasms or cramps. Vegetable glycerin and guar gum are used to counteract the drying effects of salicylate and are sufficient without additional enhancements, although other enhancements can be added (i.e., fragrances) to increase the appeal of the formulas. Lavender oil, besides its fragrance, adds additional analgesic, anti-microbial and anti-inflammatory benefits to topical formulations.

### EXAMPLE 5

Acidic topical formulas of highest concentration, particularly Formulas 5 and 6: undiluted liquid potassium salicylic acid or potassium acetylsalicylic acid used as is, is mild on mostly unbroken skin and the fastest acting on some blemishes. It can be used to relieve persistent pain, like that of shingles or deep soft tissue injury like in the heel or the ball of the foot; or it can be used to relieve persistent itches, referred to as hot spots in animals; deep lingering external pain treated repeatedly responds to concentrated, well-enhanced formulas. Since salicylates are also skin exfoliants, care should be taken to protect hands with thin disposable gloves when using repeatedly, especially when applying repeatedly to an animal. Rubbing higher dose gels externally over moderately distressed internal organs, such as over abdominal or respiratory regions, or over the throat to treat a sore throat and repeated as needed, appears to relieve the discomfort. Concurrent oral and topical use works best. Headaches, earaches, and tension pain such as in the neck, head and shoulder respond well to higher dose topical applications and over time recur less frequently.

### EXAMPLE 6

Flea and tick control in pets: Very young, ill, weakened or older animals may not be able to use products on the market due to their side effects. When severe dermatitis, most often caused by fleas and ticks, was treated with concentrated salicylate topical formulas, the dermatitis was treated as well as killing the fleas and ticks and stopping re-infestation. The back, from head to tail, was broadly treated first, followed by the rest of the body, within a few hours the repeated applications kept the skin tick and flea free. Overall skin health improved. In addition, the salicylate caused relaxation and sleep from the transdermal effects of the salicylate as well as from the salicylate ingested by the animal licking the site. The salicylate ingested by licking also worked well to relieve pain and discomfort that the animal was experiencing. Potassium salicylic acid and potassium acetylsalicylic acid topical formulations in the range of 10-20% worked best. In addition, the health and vigor of animals improved with nearly daily dosing of dry or liquid potassium salicylic Formulas 1 and 3, or occasionally from Formulas 2 and 4 of the potassium acetylsalicylic acid formulas. High oral dosing, increased gradually and monitored according to needs of the animal, was given at mealtime for most severe symptoms, particularly neurological, which responded well. The animal was able to walk straight without twisting and involuntarily turning of her head to one side and falling often as long as dosing and activity were more or less maintained. Dosing is prorated by weight for the animal on the same basis as dosing is done for humans. This dosing is also helpful when an animal needs calming, e.g., for some vet visits, for toenail cutting, shots, etc. Rubbing higher dose topical gel on paws reduces the stress of nail trims also. Chronic and long-term common ailments of dogs as they age have responded favorably to internal and external amelioration of number of ills, like neurological (such as an erratic gait and movements), gastrointestinal disorders including food poisoning of an indoor/outdoor animal, ear distress, dermatitis and arthritis. Dogs were mainly used for study purposes, but one older cat who did not respond to medical treatment for gradual renal failure, whose owner was grieving the impending loss, responded to oral dosing and became active again and continues urinating often and copiously without apparent relapse. Dosing continues ongoing. The flea and tick relief that animals get from use of the product may have broader applications to similar pest problems across species, such as that of the screw worm in cattle. Mosquito and flying insect repellence in animals and people is enhanced with regular use and with application before exposure, possibly transferable across species, but with higher concentrations needed for some species. Since the flea and tick formulation is also a skin and coat lubricant, it reduces the need for bathing and also acts as a deodorizer. An ear wash and earache relief can be produced by combining up to 2% salicylic or acetylsalicylic acid in a base of aloe vera juice and 5% vegetable glycerin, which is also suitable for humans.

### EXAMPLE 7

Dental formulation: Infections which occur from the silent breakdown of root canals or some tooth injuries require specialized attention, as an interim measure the salicylate formulas work well to relieve symptoms. A salicylate topical formulation of low concentration, e.g., under 3% is used to treat dental problems. Extra vegetable glycerin as much as 10%, is used to soothe the gums and mouth. Guar gum can be used to form a thick gel. This combination of glycerin and guar gum will adhere to gums and teeth to keep the salicylate in contact with the area being treated. The formulation can be made drier and cut into short narrow strips to put in place against gums as a poultice. The formulation can be used to maintain the health of gums and teeth as a cleaning agent for teeth leaving it on the gums long enough to ameliorate discomfort, approximately 3-5 minutes. The low acid powdered salicylates used sparingly can serve as a dentifrice which ameliorates dental problems, temporarily relieving even a toothache. Combined with dry powdered coral calcium, they make a dentifrice which relieves many minor tooth and gum irritations and keeps the mouth and gums healthy.

### EXAMPLE 8

A combination potassium salicylic acid and potassium acetylsalicylic acid dosing formula: The primary salicylates described herein, potassium salicylic acid and potassium acetylsalicylic acid, play different roles with respect to the coagulation of platelets in the blood. Salicylic acid does not play a role in thinning the blood, whereas acetylsalicylic acid does as does potassium acetylsalicylic acid. Acetylsalicylic acid is classified as a drug by the FDA (Monograph No. 94 on salicylates and analgesics), whereas salicylic acid specifically is not. One is a drug, the other appears to be emerging as more and more of a nutrient-related phyto-chemical for the present. Salicylic acid is now under intense study for its potential role in promoting health in plants and animals. Because salicylic acid and acetylsalicylic acid play opposing roles in relation to the coagulation of platelets in the blood, it is logical to expect combining potassium salicylic acid and potassium acetylsalicylic acid into one or more single formulas for using the best of each in a single therapy according to an individual's needs or for commonly occurring combinations of needs. This proposes combining the two salicylates for such needs.

Three beneficial neurological uses for salicylates: The topical formulation used alone (10% to maximum concentration for most intense locus of pain) fully ameliorated the moving shingles in one case in about one month. There has been no relapse. In another case, control is achieved over an ongoing spasticity, erratic gait and a twisting body in a neurologically impaired older but otherwise healthy dog who receives both oral and topical dosing. In a third case, chronic neurological back pain resulting from injury in a runaway skateboard accident was fully ameliorated after a period using a concentrated salicylate topical gel, following a history of unsuccessful treatment. The pain has not recurred.

Studies of salicylate's effect on plant health and vigor are increasing, suggesting more uses of the new salicylates. Of interest will be the control of soil diseases of tomatoes, etc., in warm climates which promote them.

Microbiocides for sexually transmitted diseases and possibly spermatocides might be considered for the more concentrated topical salicylates, possible highly buffered with vegetable glycerin and guar gum or essential oils.

The role of salicylates in promoting health and a sense of well-being: Ongoing use of oral and topical salicylates does much for reducing stress, prolonging productive periods within a day and increasing a sense of well-being in all activities, including resting and sleeping. Thus the result of controlling inflammation levels in the body safely may go a long way to reducing the onset and progression of degenerative ailments, especially with less than desirable lifestyles.

Topical vs. oral dosing for pain, etc.: With long-term high dose use of safer oral and topical salicylates, it becomes apparent how the salicylates affect different areas of the body needing amelioration and which modes work better. Although there is some shift from oral remedies for treating peripheral, even systemic, maladies to transdermal ones, oral dosing, especially ongoing, is still to be preferred for truly systemic ailments. Even with high dosing, however, some peripheral areas require a powerful transdermal boost at the locus of the problem, e.g., severe pain from shingles or arthritis. Respiratory and gastrointestinal ailments respond well to oral salicylates but benefit from topicals rubbed over the area of distress. A combination of topical and oral formulations usually works better than either alone but it is surprising how many otherwise apparently healthy people suffer from intense pain, like shingles or arthritis and benefit from topicals alone.

Is salicylic acid a flavonoid and therefore a nutrient? The FDA (Monograph 94 on analgesics, etc.) classifies only acetylsalicylic acid as a drug, specifically excluding other salicylates. In fact, generally, it is considered a mere phyto-chemical, awaiting elevation to the status of a nutrient by a few enthusiastic researchers trying to prove it. Two instances of heavy reformulated salicylic use suggest that salicylic acid may be more that a phyto-chemical. First, in the case of one heavy user of topical salicylic acid used alone which successfully ameliorated shingles, the mostly raw food vegan complained of a sharp drop in her ability to eat fruit which is a mainstay of her diet. This paralleled the inventor's own experience with high dosing of oral and topical salicylates. In fact, the inventor's fruit consumption of the latter had ceased entirely until this was noticed and a conscious effort to eat fruit was made, with nothing but good effects over an extended period.

1. A compound comprising a salicylic acid or acetylsalicylic acid, citric acid, and potassium hydroxide for topical or oral use.

2. A method of forming the compound as claimed in claim 1 comprising a first step of mixing said salicylic acid or said acetylsalicylic acid with said citric acid to make a salicylic acid/citric acid mixture or acetylsalicylic acid/citric acid mixture.

3. The method as claimed in claim 2 comprising said salicylic acid/citric acid mixture or said acetylsalicylic acid/citric acid mixture being dissolved in said potassium hydroxide solution to form a stabilized potassium salicylic acid solution or a stabilized potassium acetylsalicylic acid solution.

4. The method as claimed in claim 3 comprising said stabilized potassium salicylic acid or said stabilized potassium acetylsalicylic acid being further combined with additional enhancements.

5. The method as claimed in claim 4 wherein said enhancements are selected from a group of enhancements consisting of aloe vera, lavender oil, guar gum, vegetable glycerin, lemon juice, Stevia powder, coral calcium and cherry juice.

6. A compound as claimed in claim 1 where said citric acid is present in amount to make said stabilized potassium salicylic acid or said stabilized potassium acetylsalicylic acid more alkaline and to act as a preservative.

7. A compound as claimed in claim 1 comprising said citric acid being present in amount to eliminate the need for dehydration of the compound.

8. A compound as claimed in claim 1 comprising said stabilized potassium salicylic acid or stabilized potassium acetylsalicylic acid produced in oral forms selected from a group of ingestible products consisting of health drinks, soups, puddings, powders, and tablets.

9. A compound as claimed in claim 1 comprising said stabilized potassium salicylic acid or stabilized potassium acetylsalicylic acid is prepared as a topical formulation selected from a range of formulations consisting of gels, lotions, ointments, and poultices.

10. A compound as claimed in claim 1 comprising said stabilized potassium salicylic acid or said stabilized potassium acetylsalicylic acid is used for hydration, flea and tick control, dental maladies, pain relief, earaches, headaches, tension pain, arthritis, shingles, microbiocides, spermatocides, reduces stress, pesticide, deodorants, washes, lubricants, anti-burn, anti-itch, muscle spasms, and muscle cramps.

11. A method of dehydrating said stabilized potassium salicylic acid or said stabilized potassium acetylsalicylic acid for use topically or orally comprising pouring a thin layer of said stabilized potassium salicylic acid or said stabilized potassium acetylsalicylic acid onto preheated plates where said preheated plates are allowed to dry, said preheated plates are then scraped to removed the powdered potassium salicylic acid or powdered potassium acetylsalicylic acid.

12. A method of dehydrating as claimed in claim 12 wherein said preheated plates are heated to about 170° F.

13. A method of dehydrating as claimed in claim 11 wherein said dehydrating systems include fluid bed dryers, airstream systems, flash dryers, spray drier absorbers or granulators.

14. A method of blending topical formulation as claimed in claim 1 comprising diluting said stabilized potassium salicylic acid solution or said stabilized potassium acetylsalicylic acid solution with water to cool said stabilized potassium salicylic acid solution or said stabilized potassium acetylsalicylic acid solution and blending with cold pressed aloe vera.

15. A method of blending topical formulation as claimed in claim 15 comprising further combining said stabilized potassium salicylic acid solution or said stabilized potassium acetylsalicylic acid solution and said aloe vera with additional enhancements to form a blended topical formulation.

16. A method of blending topical formulation as claimed in claim 16 comprising said enhancements are selected from a group of enhancements consisting of lavender, guar gum, and vegetable glycerin.

17. The compound of claim 1 including additional enhancements selected from a group of enhancements consisting of aloe vera, lavender oil, guar gum, vegetable glycerin, lemon juice, Stevia powder, coral calcium and cherry juice.
